(19) United States
(12) Patent Application Publication
(10) Pub. No.: US 2013/0085481 A1
(43) Pub. Date: Apr. 4, 2013

(54) OPHTHALMOLOGY APPLIANCE FOR PHOTOCOAGULATION OR PHOTOTHERAPY, AND METHOD FOR OPERATING SUCH AN APPLIANCE

(71) Applicants: Manfred Dick, Gefell (DE); René Denner, Reisdorf (DE); Gerald Kunath-Fandrei, Jena (DE)

(72) Inventors: Manfred Dick, Gefell (DE); René Denner, Reisdorf (DE); Gerald Kunath-Fandrei, Jena (DE)

(73) Assignee: CARL ZEISS MEDITEC AG, Jena (DE)

(21) Appl. No.: 13/702,044

(22) PCT Filed: May 28, 2011
(86) PCT No.: PCT/EP2011/002648

§ 371 Date: Dec. 4, 2012

(30) Foreign Application Priority Data
Jun. 4, 2010 (DE) 102010022760.9

(51) Int. Cl. A61F 9/008; A61F 9/007

(52) U.S. Cl. CPC A61F 9/00821; A61F 9/007
USPC 606/4

(57) ABSTRACT

A device intended to permit photocoagulation or phototherapy at low cost and in a shorter time. For this purpose, an ophthalmology appliance includes a radiation source having several discrete individual emitters. The therapy beam path leading from the radiation source to the treatment area projects an image of at least respective portions of different individual emitters simultaneously onto surfaces spaced apart from each other in the treatment area. This permits simultaneous generation of several coagulation foci and dispenses with the need for electromechanical beam-deflecting units, which permits a shorter treatment time.

### PRIORITY CLAIM

The present application is a National Phase entry of PCT Application No. PCT/EP2011/002648, filed May 28, 2011, which claims priority from German Application No. 10 2010 022 760.9, filed Jun. 4, 2010, the disclosures of which are hereby incorporated by reference herein in their entirety.

### FIELD OF THE INVENTION

The invention relates to an ophthalmological device having a radiation source for photocoagulation or phototherapy of tissue, particularly a retina of an eye, a therapy beam path extending from the radiation source to a treatment region, and an observation beam path.

### BACKGROUND

Light coagulation or photocoagulation is a therapy in use since 1949 for different diseases of the retina, for example, retinal detachment. While xenon high-pressure lamps and even sunlight were initially used, lasers which emit continuous waves (cw) are normally used today as light source. Photocoagulation is then called laser coagulation.

At first, the eye is placed in the treatment region, so the therapy beam path can be focused on the retina, and a contact glass is placed on the eye. With a so-called pilot beam with non-coagulating radiation power (hereinafter also called luminous power), the target region to be treated is visually marked through the contact glass on the retina. The pilot beam can be coupled into the therapy beam path or generated with the radiation source, which is provided for photocoagulation, using a beam attenuator or power modulator. After visual examination of the target region with the pilot beam, the operator can manually trigger the treatment process. Then the marked region is irradiated with coagulating luminous power. The applied light is, e.g., absorbed in the retinal pigment epithelium (RPE), a layer in the retina which has a dark pigment (particularly melanin), and a coagulation spot is thus generated. Through thermal conduction, the adjacent tissue is also heated, causing it to become scarred.

The main area of photocoagulation is the shifting of the metabolism onto the still healthy regions of the retina from atrophying diseased tissue. Photocoagulation can also stimulate biochemical cofactors. For example, the progress of diabetic retinopathy can be significantly slowed down or halted. In case of holes in the macula or early retinal detachments, the scarring can be used to attach the retina to the underlying layer of the ocular bulb, the choroid.

Depending on the effect to be achieved, coagulation spots are placed locally in the outer layers or globally in the entire retina during treatment. For example, during several temporally spaced treatment sessions, up to approximately 3000 single spots are successively generated, each with an irradiation duration of, e.g., 100 ms at an irradiation energy of, e.g., 100 mW. Depending on the target region of the coagulation spots to be placed, different spectral ranges are used for irradiation. Green light is used most commonly. Yellow light is used for the area of the macula and for atrophying of retinal blood vessels while red light is used for particularly great penetration depths, e.g., for the treatment of vessels of the choroid.

Conventional photocoagulation is disadvantageous due to high costs for a laser beam source with a comparatively high beam quality in order to be able to couple the radiation into an optical fiber with a typical diameter of 50 μm. As a result, the radiation is transported to the actual applicators, e.g., a laser slit lamp, a fundus camera, or a headworn ophthalmoscope, in order to be reflected into the observation beam path. The laser device typically has a cumbersome size of approximately 20×30×15 cm³. In addition, the treatment is relatively time-consuming, usually 20 to 30 minutes, due to the manual generation of a large number of individual coagulation spots, which is uncomfortable for the patient.

More recently, the improvement of the successive manual target search with scanning, electromechanical beam-deflecting devices (scanners) in the therapy beam path has been described, for example, in WO 2005/122872 A2 and WO 2007/035855 A2. These allow for the automatic generation of fields of coagulation spots which are also called coagulation patterns. The laser beam is incrementally positioned using the beam-deflecting device and the desired laser power is then provided for the correct position.

This type of laser coagulation is disadvantageous because the sequence of successive scanning and irradiation of a coagulation pattern can last up to 0.5 sec during which the patient's eye can move, resulting in a possible misalignment within the pattern. Moreover, the system of the mostly electromechanical beam-deflecting devices is costly and high-maintenance due to signs of wear.

### SUMMARY OF THE INVENTION

The problem addressed by the invention is that of improving a device of the type initially described in such a way that photocoagulation or phototherapy is possible at low expenditures in a shorter period of time.

The problem is solved with a device which has the features discussed herein, and a method with the features discussed below. According to the invention, the radiation source has a plurality of disjoined single emitters and the therapy beam path projects at least certain segments of different single emitters simultaneously onto surfaces in the treatment region which are spaced apart from each another. According to the invention, single emitters are separate light sources which can be controlled jointly, in groups or individually. Each of the surfaces which are spaced apart from each another is a potential coagulation spot (“coagulation spot surface”). Due to the distances between the irradiated surfaces, the potential coagulation spots are disjoined. The single emitters are not necessarily spaced apart from each other but can also abut each other. The distances of the surfaces can be generated, e.g., through optical elements in the therapy beam path, such as apertures. The single emitters do not necessarily have to be lasers, instead, individual or all single emitters can be light-emitting diodes (LED) without stimulated emission and without amplification.

The invention allows for the simultaneous generation of a plurality of coagulation spots, resulting in a shorter treatment duration when compared to conventional procedures. The spatial structure of the relative arrangement of the single emitters to each other essentially predetermines the coagulation pattern to be generated, wherein individual emitters can remain deactivated, and so the corresponding potential coagulation spots are not coagulated. With respect to the prior art, no elaborate electromechanical beam-deflecting devices are required due to disjoined single emitters which can simultaneously generate disjoined coagulation spots. An irradiation relief can be generated through an irradiation power which is variably adjustable beyond the number of single emitters and/or through different spectral ranges beyond the number of single emitters. For example, the irradiation power can be adjusted using individual power control circuits for the single emitters. With the same single emitters, it is also possible to first emit non-coagulating pilot beams (“pilot beam mode”) and subsequently emit coagulating beams (“coagulation mode”). In addition to the simultaneous spatial forming of the coagulation pattern, it is also possible to temporally structure and form the emission times of the coagulation spot surfaces and/or the coagulation pattern.

For example, in a special embodiment, in which the single emitters are arranged two-dimensionally distributed, a series of sequentially irradiated, disjoined lines can be coagulated, wherein the operator must confirm the execution of the irradiation prior to every line using an operating procedure. A line consists of a plurality of coagulation spot surfaces, and due to the distances between said surfaces, the line is thus discontinuous. The length of the line is variable based on the single emitters used. After the first executed coagulation of a first line in coagulation mode, the second potential line is displayed in this embodiment in the pilot beam mode and a confirmation is determined by the operator. If the operator approves, he/she triggers the coagulation mode of said second line and a potential third line is displayed immediately until the options of the two-dimensional emitter arrangement are exhausted. Now the operator can establish a new treatment area on the retina via the observation beam path in order to place widespread coagulation spots. For example, the emitters can be arranged in a rectangular matrix of straight rows and columns, wherein every line corresponds to a respective row of the matrix or at least a part thereof. The lines do not necessarily have to be straight, curved lines are also conceivable with straight rows and columns. The different lines do not have to congruent because it is possible that they have different radii of curvature and/or different lengths. Due to the individual responsiveness of the single emitters, any type of form can be predetermined. Alternatively to a rectangular row/column arrangement, the single emitters can also be arranged along curved lines or irregular curves.

The single emitters are preferably designed as semiconductor diodes, particularly as laser diodes. In particular, they can be arranged in the form of at least one diode matrix (diode array) on a joint substrate. With the parallel offset arrangement of a plurality of substrates, a correspondingly greater number of single emitters can be provided. Such radiation sources are commercially available, e.g., in the form of vertical-cavity surface-emitting lasers (VCSEL). They have the advantage of a narrow beam concentration, and therefore special ancillary optics for collimating the laser radiation on the retina are not required. Due to its intrinsic concentration, a single VCSEL used as single emitter, e.g., can generate a single coagulation spot surface on the retina with a diameter of 200 μm. A VCSEL matrix, e.g., can have 5×5 laser diodes as single emitters. In addition, diode laser matrices, which are called LED laser arrays, are known, e.g., from US 2006/0215950 A1. For example, they can comprise hundreds of single emitters. When compared to gas or solid-state lasers, semiconductor lasers have a better thermal efficiency, so they can be operated in the immediate vicinity of the patient and the operator without causing harm. In particular, the coupling into the device is possible at close proximity to the eye. By controlling the radiation power, LED laser arrays can be used, according to the invention, as light-emitting diode arrays below the laser threshold.

It is possible, but not mandatory, to arrange several optics side by side in the therapy beam path for projecting the single emitters into the treatment region. E.g., these can be microlenses, particularly in the form of an arrangement which corresponds to the arrangement of the single emitters but is scaled on the basis of the distance from the single emitters. For example, the optics can be arranged in the shape of a matrix. The optics are preferably used for collimating the light radiation and/or improving the beam quality and/or defining the surface sizes and the beam profiles. With the use of one optics (microlens), one or more single emitters can be projected simultaneously: Embodiments are preferred in which at least one of the optics is arranged upstream of a corresponding group of single emitters such that it acts on the light emitted from said single emitters, thus projecting them simultaneously into the treatment region.

For example, the device can be designed such that different single emitters of the group emit different, particularly disjoined spectral ranges. This allows for the irradiation of a coagulation spot surface with different colors, depending on the treatment. For example, a plurality or even all groups of single emitters are designed in such a spectrally variable way. The single emitters of a respective group can also be considered to be sub-emitters of a single emitter, wherein the entire group must be considered to be the actual single emitter.

In an example embodiment, the device has a plurality of groups of single emitters, wherein each of these groups is arranged upstream of a corresponding joint optics (for projecting the respective group into the treatment region) and at least one single emitter of each group is designed to emit non-coagulating light, and at least one other single emitter of each group, particularly at least two other single emitters with differing emission spectral ranges, are designed to emit coagulating light. This allows for simultaneously generating pilot beams for the corresponding coagulation spot surfaces, wherein no elaborate beam attenuators are required. For example, the only non-coagulating single emitter of an example group emits with a pure illumination light power of 1.5 mW in the red spectral range as opposed to a coagulation light power of 100 mW of three coagulating green single emitters of the group. In addition or alternatively to a low power, the pilot beams can have an exclusively non-coagulating color. The coagulating single emitters of a/every group can alternatively emit differing spectral ranges, e.g., red, yellow, and green. Expediently, they can be controlled independently from each other.

According to an example embodiment of the invention, the light-emitting diodes or laser diode arrays are optionally composed of only one type of emitter, e.g., with a central wavelength of 532 nm or 580 nm or 630 nm, or of a mixture of different types of emitters (with different central wavelengths) and have optionally one microlens each downstream of each single emitter, or a microlens combines a respective group of a plurality of single emitters to an irradiated surface in order to achieve a power increase in the surface. In the combined arrangement, e.g., a red light-emitting diode can act as non-coagulating pilot beam source and three additional green light-emitting diodes can act as coagulating therapy beam sources in order to allow for a precisely positioned superimposition and display of the potential coagulation spot surfaces. However, for displaying the potential coagulation spot surfaces, the actual therapy radiation emitters can also be used in a low-power mode (pilot beam mode), their control system permitting.

Alternatively to adjusting the single emitters to a coagulating irradiation power which irreversibly destroys tissue (coagulation mode) and to adjusting to a non-coagulating power in the pilot beam mode which is merely an optical illumination for displaying a subsequent treatment point without impact on the tissue, some of the single emitters or the entire radiation source can, according to the invention, provide a third power level between the non-coagulating irradiation power of the pilot beam mode and the coagulation mode in such a way that the tissue is stimulated below the coagulation threshold (hereinafter called laser therapy mode). For example, interpolation of a proper subset of single emitters of a group can create the third power level, when all single emitters of the group jointly emit, in total, a coagulating luminous power.

In addition to the clocked continuous radiation emission for the coagulation mode, the radiation source advantageously provides a pulsed radiation emission for the laser therapy mode with pulse lengths from the femtosecond range to the millisecond range, particularly from the nanosecond to the microsecond range, and for which the single emitters must be designed as laser diodes. In this pulsed operating mode of the laser therapy mode, the device, according to the invention, also allows a selective retina therapy (SRT) or a retinal regeneration therapy (2RT) which generate a photoacoustic effect for the therapy of the retina, particularly in the retinal pigment epithelium. According to the invention, the laser therapy mode can be used alternatively to the coagulation mode described below.

According to the invention, a non-coagulating single emitter has a control device which controls the radiation power emitted by said emitter such that the radiation is absorbed and/or scattered and/or reflected on the retina without coagulation. The controlled radiation power can be preset or adjustable. With adjustable radiation power, the respective single emitter can emit radiation optionally with pilot beam power, laser therapy power or coagulation power. The control device can be designed, e.g., as electronic circuit.

After the introduction of a photoactivable active agent, particularly a monoactive benzoporphyrin derivative, e.g., C₄₁H₄₂N₄O₈, in the treatment region, the active agent can be activated selectively parallel in a cross-section of any given form through simultaneous activation of a plurality of or all single emitters of the radiation source, according to am embodiment of the invention, particularly, exclusively a plurality of or all non-coagulating emitters.

For example, the pilot beam single emitters can be used in this manner in the red range as an photodynamic therapy irradiation matrix (PDT irradiation matrix) for combination therapy. In conjunction with an imaging system including a motion-tracking algorithm, it is possible to destroy pathological neovascular blood vessels while “normal” vessels localized in the immediate vicinity remain undamaged because only those tissue portions are therapeutically affected which have absorbed the active agent and have been activatingly irradiated.

In addition, the ophthalmological device can also be used as a microperimeter, for example in the visible spectral range and in another example in the blue or yellow wavelength range (so-called blue-yellow perimetry) for a quick analysis of the field of vision with 2D subpatterns. In such case, the low-power pilot beam mode must be used.

The emission duration and/or emission power of the single emitters (particularly light-emitting diodes or laser diodes) can be constant identical, or optionally constant different, or vary differently. With regard to emission duration and/or emission power, the single emitters can preferably be controlled independently from each other. This allows for greatest flexibility during planning and execution of the surgery.

For example, a plurality of groups of single emitters or at least one proper subset of single emitters can be designed for the emission of white light with a non-coagulating illumination light power. This provides both the pilot and therapy radiation from single emitters but also a retinal illumination of the surgical environment. These single emitters, e.g., can be white-light-emitting diodes or a subset of RGB LEDs. Particularly in a matrix-shaped arrangement of the single emitters, the white-light emitting single emitters or subsets can be arranged in the outer fields of the single-emitter matrix. Optionally, the white-light emitting single emitters or subsets can be projected directly into the treatment region for homogenous illumination of the surgical field. For this purpose, the therapy beam path in the region of these single emitters can be free of optics (single optics or group optics). The white-light illumination can advantageously be controlled temporally and spatially structured by a control unit, e.g., in order to generate different light slits in the case of a slit lamp. In addition to this variation of straight light slits of varying size and orientation without movable parts, curved slits or free-formed slits are also possible.

A linear matrix-shaped arrangement of the single emitters allows particularly for the use of line-shaped treatment “combs” which do not necessarily require the addition of a digital imaging system and the adjustment of a planar treatment emitter matrix on this image. In addition, a one-dimensionally spatially resolving line detector with an automated offset/shift of a line to be irradiated can be used in order to carry out a linear (image) coagulation with optical feedback. Single emitters can be automatically selected for a subsequent line-shaped irradiation. In a particular embodiment, the emitters can be arranged exclusively along at least one straight line, wherein a detection beam path projects the respective surfaces of the treatment region onto at least one row of a plurality of detector elements. E.g., there can be a number of parallel straight lines, when the single emitters are arranged as rectangular matrix in rows and columns. This allows for the image-supported determination of information for a selection of the single emitters for the subsequent coagulation line during sequential line-shaped coagulation. For example, this might be necessary in order to exclude healthy tissue from a line-shaped irradiation when healthy tissue in the region of the next irradiation is determined with the detector elements. The single emitter(s) in the region of the healthy tissue can thus remain already deactivated in the pilot beam mode. However, using a special operating element, the operator can also force the irradiation of said region. Instead of a line detector, a two-dimensionally spatially resolving matrix detector can also be used, the two-dimensional image of which is analyzed only for certain regions.

For example, the optics are microlenses, particularly in the form of a coherent matrix. Microlenses are commercially available at low cost. A microlens matrix can be placed directly onto the radiation source and particularly lie flush against said radiation source.

The therapy beam path is preferably free of mirrors, which are movable for scanning of the treatment region, and free of lenses, which are movable for scanning of the treatment region, and free of fiber-optic cables. The omission of movable optical elements such as mirrors and lenses ensures that the device is largely maintenance-free and, including the omission of fiber-optic cables, also keeps expenditures and maintenance costs low. Moreover, the omission of fiber-optic components significantly additionally decreases radiation energy losses. The small dimensions (in the cm³ range) and low heat loss (only approximately 35 W at 2 to 5 W optical power) of a light-emitting and laser diode array as structured radiation source make said omission possible.

Advantageously, a zoom optics can be arranged in the therapy beam path in order to allow for a change of the imaging scale and thus the size and distance of the coagulation spot surfaces.

The single emitters and particularly the downstream optics are preferably designed such that a balance ratio of a distance between two adjacent coagulation spot surfaces in the treatment region and a diameter of one of the surfaces is between one and two. This device feature allows for a most efficient treatment with the lowest possible side effects.

Regardless of particularly simultaneous changes of the surface sizes and surface distances, the gaps between the individual coagulation spot surfaces, e.g., can be changed for a specific surface size by leaving the subsets of the single emitters deactivated. For example, it is possible to irradiate the same surface size at approximately twice the surface distance, when only every other single emitter is activated. Alternatively or additionally, the gaps can be changed, e.g., using digital micro-mirror devices (DMD).

Embodiments can have a control unit for successive activation of different, particularly disjoined subsets of all single emitters, particularly with at least two elements in each subset, and particularly with congruent envelopes of said subsets. Expediently, every subset is deactivated before the next subset is activated. This sequence allows for the simulation of a scan movement. Preferably, the single emitters of one subset are arranged line-shaped, wherein the lines thus formed run parallel, so the envelope is a rectangle. This allows for a quick line-shaped scan without an (electro)mechanical beam-deflecting device. In particular, the control unit can activate exclusively white-light emitting single emitters at such a line-shaped scan in order to substitute the movement of a slit lamp. Therefore, a white ambient illumination integrated in the radiation source similar to a digital slit lamp allows for the omission of the conventional slit illumination.

Expediently, the therapy beam path is coupled into the observation beam path of the ophthalmological device. This allows for high accuracy when the conformity of the momentary treatment region and desired target region is verified. The therapy beam path can be coupled into the observation beam path, e.g., via a beam splitter which can be rigid or optionally movable into the observation beam path.

In addition or alternatively, the radiation source can be energized by a battery and/or an accumulator such that the ophthalmological device can be operated independently of the main power supply. This allows for a significantly greater freedom of movement for the operator since no power cord is required. In particular, the risk of medical errors in the periphery of the freedom of movement, as is known from the prior art, is avoided.

The device according to the invention can be called a coagulator. In particular, it can refer to a slit lamp, a fundus camera or a headworn ophthalmoscope.

The invention also comprises a method for operating a device, designed according to the above description, wherein the following steps are carried out, particularly by a control unit:

Activation of a single emitter with non-coagulating radiation power in every group of single emitters which is jointly projected by a joint optics (for simultaneous pilot beam emission),
identification of a signal for coagulation,
activation of at least one single emitter with coagulating radiation power in each of the groups (for generating coagulation spots).

The advantages of this method are the short treatment duration and the constant relative position of the irradiated coagulation spot surfaces. The emitters activated for the pilot beam emission and the emitters activated for coagulation can be identical by operating them with correspondingly different electric power.

The invention comprises a further method for operating a device, designed according to the above description, wherein the following steps are carried out:
Activation of a plurality of single emitters for irradiating along a first line with non-coagulating radiation power,
identification of a signal for coagulation,
activation of a plurality of single emitters for irradiating along the first line with coagulating radiation power, and
repetition of the previous steps with a plurality of other single emitters for irradiating along a second line which is disjoined from the first line.

The advantages of this method are the short treatment duration and the constant relative position of the irradiated coagulation spot surfaces. The emitters activated at a respective line for the pilot beam emission and the emitters activated for coagulation can be identical by operating them with correspondingly different electric power.

The invention also comprises a control unit for a device, designed according to the above description, and a computer program for such a control unit which is designed for executing the method according to the invention. Expediently, the computer program is stored in a data storage device and comprises:
A software module for activating a single emitter with non-coagulating radiation power in every group of single emitters which is jointly projected by a joint optics for simultaneous pilot beam emission,
a software module for identifying a signal for coagulation, and
a software module for activating at least one single emitter with coagulating radiation power in each of the groups for coagulation.

This can refer to different software modules or one and the same software module for all method steps.

In an alternative manifestation, the computer program can comprise:

A software module for activating a plurality of single emitters for irradiating along a first line with non-coagulating radiation power,

a software module for identifying a signal for coagulation, and

a software module for activating a plurality of single emitters for irradiating along the first line with coagulating radiation power, wherein the computer program is designed for repeated activation of the aforementioned software modules with a plurality of other single emitters for irradiating along a second line which is disjoined from the first line.

Once again, this can refer to different software modules or one and the same software module for all method steps.

In the following, the invention is further described in terms of embodiments.

### BRIEF DESCRIPTION OF THE DRAWINGS

The drawings depict:

FIGS. 1 and 2: Monochromatic light-emitting diode arrays;

FIGS. 3 and 4: Dichromatic light-emitting diode arrays;

FIGS. 5 and 6: Dichromatic light-emitting diode arrays with integrated ambient illumination;

FIG. 7: A monochromatic light-emitting diode array with additional optics;

FIG. 8: A slit lamp with external coagulator;

FIG. 9: A slit lamp with internal coagulator;

FIG. 10: A headworn ophthalmoscope with conventional ambient illumination; and

FIG. 11: A headworn ophthalmoscope with integrated ambient illumination.

### DETAILED DESCRIPTION

Corresponding components have the same reference signs in all drawings.

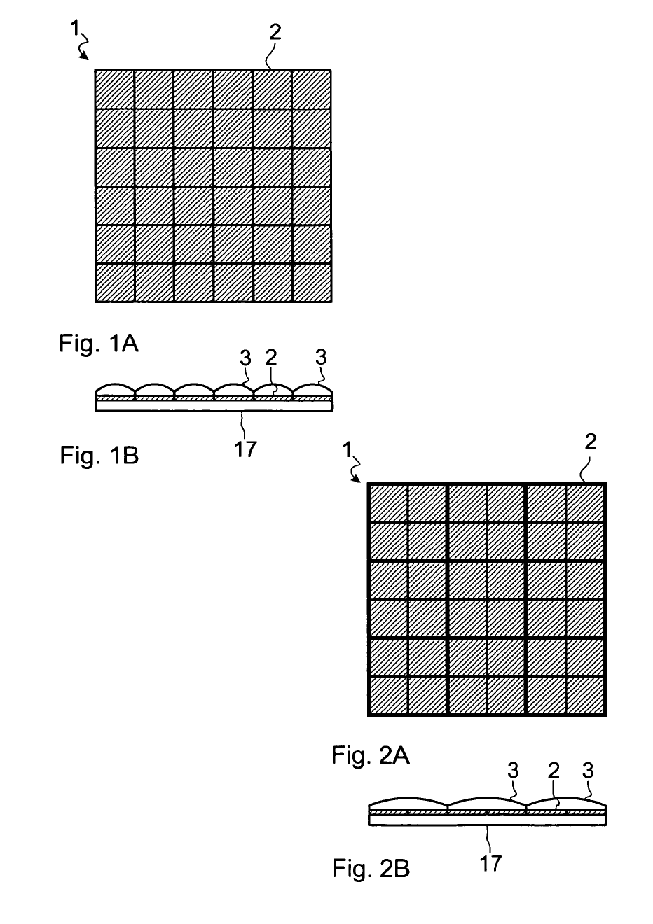

FIG. 1 schematically depicts a light-emitting diode array as structured radiation source 1 with, e.g., 6×6 single emitters 2 which emit light in a spectral range of, e.g. 511 nm to 553 nm (central wavelength 532 nm), identical for all emitters 2. According to the invention, the radiation source 1 can be used as main component of a coagulator (not depicted). The coagulator can be part of a more complex device (not depicted). The therapy beam path and the treatment region are not depicted for reasons of simplification.

The single emitters 2 are semiconductor light-emitting diodes which are arranged equidistantly on a joint substrate 17. In detailed FIG. 1A, the matrix 1 is depicted as topview, detailed FIG. 1B shows the profile of said matrix. In the profile, the 36 micro-optics 3 which are directly connected to the matrix substrate 17 are shown in an example form as an integrally designed microlens matrix. Every single emitter 2 is thus provided with its own micro-optics 3. With such an arrangement, e.g., 36 coagulation spot surfaces, which are spaced apart from each other, can be irradiated and thus a corresponding number of coagulation spots be generated. The single emitters 2 are individually controllable, and therefore any type of combinations of coagulation spot surfaces can be generated. Instead of light-emitting diodes, the single emitters 2 can be designed as laser diodes.

In alternative embodiments, the matrix 1 can have any number of m×n (m, n natural numbers) of single emitters 2 (and a corresponding number of optics 3). This is possible, e.g., by arranging a plurality of substrates flush with each other, wherein each substrate carries its own light-emitting diode array.

FIG. 2 shows a light-emitting diode array identical with the one in FIG. 1 but having only nine micro-optics 3 which are larger than the micro-optics in FIG. 1. Every micro-optics 3 acts on the light in a respective group of four single emitters 2 which are depicted with bold lines. This allows for the irradiation of maximally only nine coagulation spot surfaces which are spaced apart from each other, but the irradiation power of each group can be selected incrementally due to the option of activating zero to four single emitters of a group. Detailed FIG. 2B shows the side view.

Using an adjustable control circuit for the diode current of the single emitters 2, the irradiation power is also infinitely adjustable.

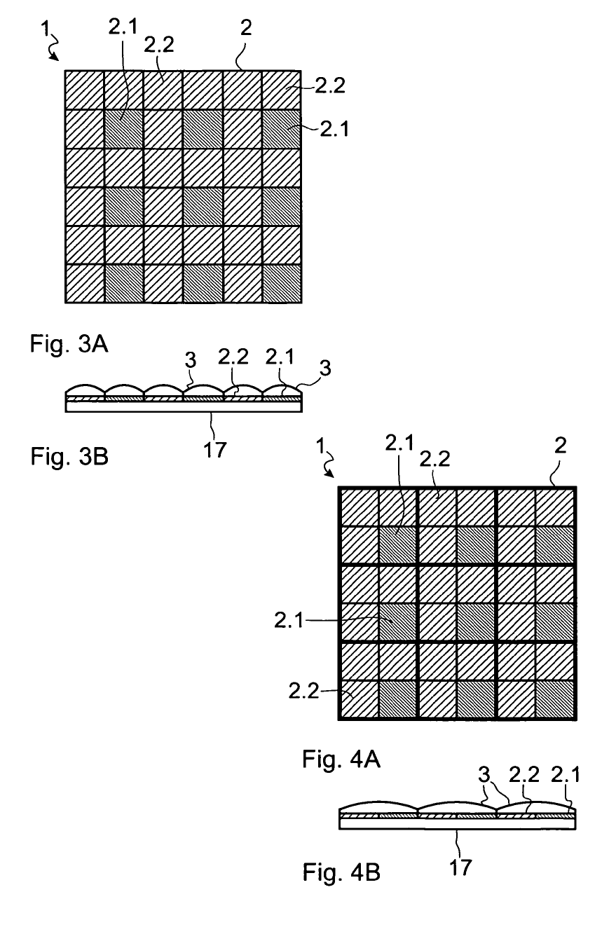

FIG. 3 shows a light-emitting diode array 1 with, e.g., 6×6 single emitters 2 of two different spectral ranges. The single emitters 2.1, which constitute a quarter of the total amount of single emitters 2, emit light in a spectral range of, e.g. 600 nm to 630 nm with a non-coagulating radiation power of, e.g., 1.5 mW per single emitter 2. These single emitters 2 are evenly distributed over the matrix 1 in a regular arrangement. The remaining single emitters 2.2 emit light in a spectral range of, e.g. 430 nm to 460 nm with a coagulating radiation power of, e.g., 100 mW. Detailed FIG. 3B shows the side view.

Instead of light-emitting diodes, the single emitters 2 can be designed as laser diodes. Particularly, only the coagulating single emitters 2.2 can be designed as laser diodes while the non-coagulating single emitters 2.1 are designed as light-emitting diodes.

FIG. 4 shows a light-emitting diode array 1 identical with the one in FIG. 3, wherein four single emitters 2 each are combined with a joint optics 3 to a group with bold lines. Detailed FIG. 4B shows the side view. The groups are formed such that each optics 3 projects one of the non-coagulating single emitters 2.1 at a time. Coagulation is possible in nine surfaces with three intensity steps each (1 to three coagulating single emitters 2.2 which are independently controllable. When all coagulating emitters 2.2 of a group (preferably all groups) are deactivated, the respective non-coagulating single emitter 2.1 of the group(s) can be used for pilot beam emission(s).

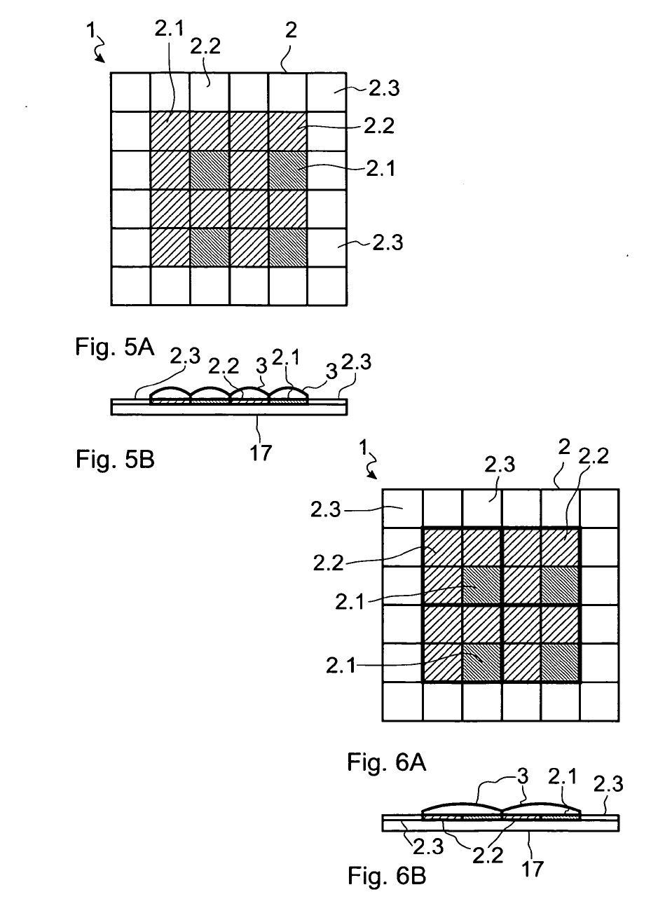

FIG. 5 shows a light-emitting diode array 1 with, e.g. 6×6 single emitters 2 of three different spectral ranges. The spectral ranges of the coagulating single emitters 2.2 and the non-coagulating single emitters 2.1 are disjoined while the single emitters 2.3 in a circular arrangement on the outer edge emit white light with non-coagulating radiation power as ambient illumination. The side view depicted in detailed FIG. 5B shows that no optics 3 are arranged downstream of these single emitters 2.3, so the entire treatment region is illuminated. Using the coagulating single emitters 2.2, this arrangement allows for the irradiation of twelve coagulation spot surfaces, which are spaced apart from each other, in the treatment region. The non-coagulating single emitters 2.2 are used to depict four pilot surfaces in the treatment region.

Expediently, the ambient illumination from the single emitters 2.3 is deactivated during this process.

Instead of the circular arrangement on the outer edge of the matrix 1, each group, e.g., can be enclosed all around by ambient-illuminating single emitters 2.3. Expediently, the single emitters 2.3 are also free of optics 3.

FIG. 6 shows a light-emitting diode array 1 identical with the one in FIG. 5, wherein a plurality of single emitters 2, in this case, e.g., one non-coagulating single emitter 2.1 and three coagulating single emitters 2.2, which are independently controllable, are combined to a group with a corresponding joint optics 3. Using the single emitters 2.2, this arrangement allows for the simultaneous irradiation of four coagulation spot surfaces in the treatment region with variable power. Alternatively or simultaneously, four pilot beams can be generated simultaneously using the single emitters 2.1. Alternatively or simultaneously, the treatment region can be ambiently illuminated using the single emitters 2.3.

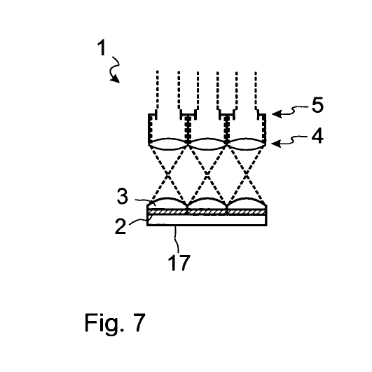

FIG. 7 shows a schematic side view of a radiation source 1 with, e.g., 3×3 laser diodes 2 including optics 3 on a substrate 17, and an additional microlens system 4 and a downstream micro-aperture system 5 for improving the imaging quality. This arrangement, e.g., decreases crosstalk between the sub-beam paths of the single emitters 2.

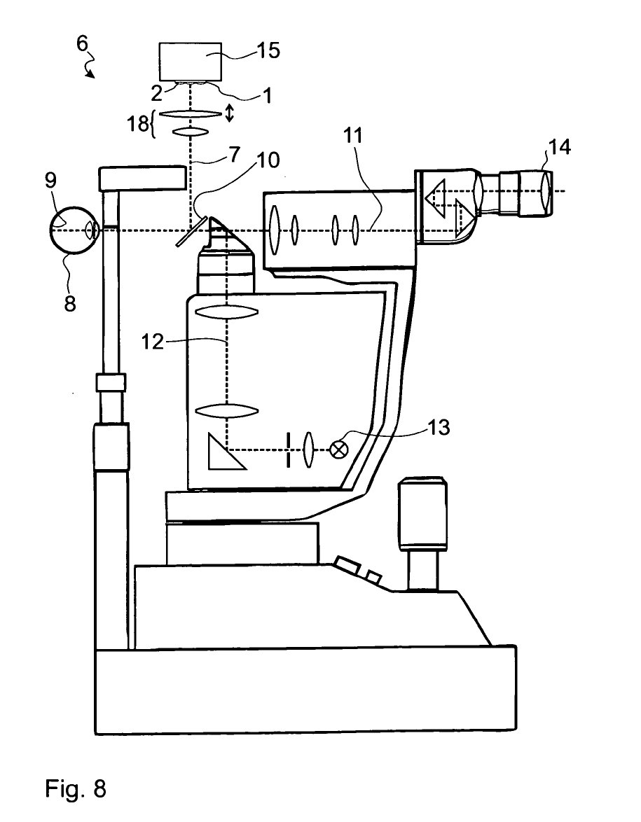

FIG. 8 schematically depicts the device 6 as a slit lamp which is known from the prior art, having a light-emitting diode array according to the invention, e.g., according to FIG. 4, as structured external radiation source 1. The therapy beam path 7 extends from the radiation source 1, i.e., from the single emitters 2, to the retina of the eye 8, which constitutes the treatment region 9. It is coupled into the observation beam path 11 as link via a rigid beam splitter 10. An illumination beam path 12 with a mercury lamp as separate light source 13 is used for the slit-shaped illumination of the treatment region 9. Through eyepieces 14, an operator can visually observe the points of impact of the nine pilot beams of the non-coagulating single emitters 2.1 in the treatment region 9 and trigger a simultaneous photocoagulation with the coagulating single emitters 2.2 in the corresponding nine surfaces. A zoom optics 18 is arranged in the therapy beam path 7 in order to variably adjust the imaging properties of the beam path.

Due to the type of coupling via a connecting element 10, detachably fastened to the device 6, the radiation source 1 can be separated from the device 6 and used elsewhere.

Instead of eyepieces 14, digital cameras, e.g., can be arranged in the observation beam path 11. Instead of a binocular arrangement, a single eyepiece, e.g., a single digital camera can be provided. Digitally recorded images, e.g., can be depicted in real time on a connected control unit or its visual display unit. For this purpose, the digital images can be transmitted, particularly wirelessly, to the control unit in order to allow for great freedom of movement. With two separate digital cameras, a three-dimensional view can be determined stereoscopically and depicted on a suitable visual display unit.

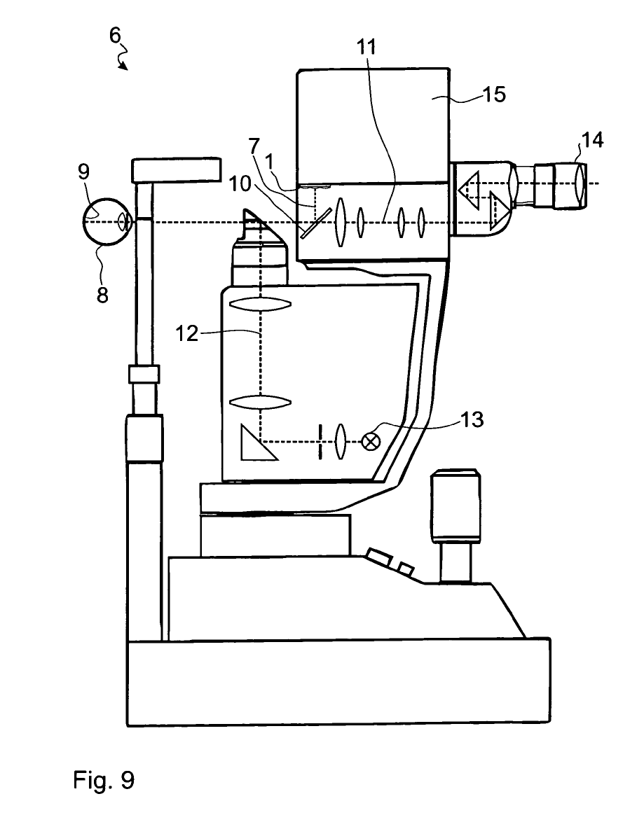

FIG. 9 shows a slit lamp as device 6, which substantially corresponds with the slit lamp shown in FIG. 8, wherein the structured radiation source 1 and a corresponding control unit 15 are permanently integrated in the device 6. The operating elements of the control unit 1 can thus be ergonomically integrated in the control concept of the slit lamp 6.

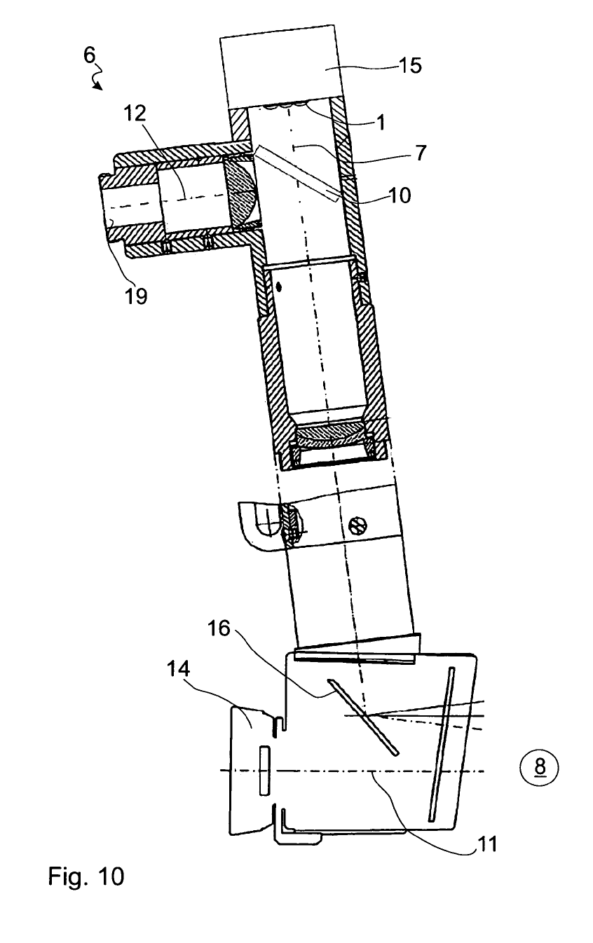

FIG. 10 shows a cross-section of a headworn ophthalmoscope, which is already known, as device 6, having both an end face 19 for an external light source (not depicted) for conventional ambient illumination and, according to the invention, a structured radiation source 1 for photocoagulation along an illumination beam path 12. The illumination beam path 12 and the therapy beam path 7 are coupled using a beam splitter 10 prior to being coupled into the observation beam path 11 via a mirror 16. The radiation source 1 is mounted on its control unit 15 which also contains the power supply (not depicted) of the radiation source 1.

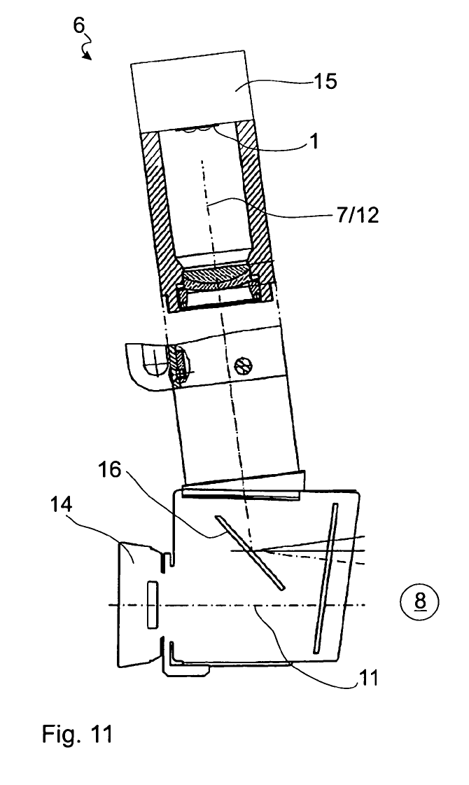

FIG. 11 shows a headworn ophthalmoscope as device 6, in which a radiation source 1, e.g., according to FIG. 6, with white-light emitting, non-coagulating single emitters 2.3 is used for both photocoagulation and ambient illumination. No connection and optical fiber to an external light source are required, which significantly improves the operator's freedom of movement. The control unit 15 merely requires a flexibly designable electrical supply cable. Otherwise, the device 6 corresponds to the device shown in FIG. 10. In alternative embodiments (not depicted), it is also possible to provide the radiation source 1 with electric energy using an accumulator for maximizing the operator's freedom of movement.

For example, the depicted devices 6 can be used for sequential line-type irradiation of the retina, wherein initially the first line to be irradiated is marked with pilot beams by activating a respective subset of the non-coagulating single emitters 2.1. Through actuation of an operating element of the device 6, the operator triggers the signal for coagulation, and coagulation spot surfaces are subsequently irradiated with coagulating luminous power along the indicated first line. Immediately after said irradiation, the control unit 15 automatically visually marks, e.g., an adjacent second line with pilot beams using other single emitters 2.1 and anticipates the next triggering of the signal for coagulation.

If the first and second line and also further lines are disjoined, the respective next line can already be automatically marked with pilot beams while the previous line is still irradiated coagulatingly. This can expedite the treatment.

Laser diode arrays instead of light-emitting diode arrays can alternatively be used in all embodiments.

### REFERENCE SIGNS

1 Radiation source
2 Single emitters

### 3 Micro-optics

4 Microlens system
5 Micro-aperture system

### 6 Device

7 Therapy beam path

### 8 Eye

9 Treatment region
10 Beam splitter
11 Observation beam path
12 Illumination beam path
13 Light source

### 14 Eyepiece

15 Control unit

### 16 Mirror

### 17 Substrate

18 Zoom optics

1-17. (canceled)

18. An ophthalmological device, comprising:
a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other.

19. The device according to claim 18, wherein the single emitters comprise semiconductor diodes.

20. The device according to claim 19, wherein the single emitters comprise laser diodes.

21. The device according to claim 19, wherein the single emitters comprise at least one diode array on a joint substrate.

22. The device according to claim 18, further comprising a plurality of optics that project the single emitters into the treatment region, the optics being arranged next to each other in the therapy beam path.

23. The device according to claim 19, further comprising a plurality of optics that project the single emitters into the treatment region, the optics being arranged next to each other in the therapy beam path.

24. The device according to claim 22, wherein at least one of the optics is arranged downstream of a respective group of a plurality of single emitters such that it acts on the light emitted from said single emitters.

25. The device according to claim 24, wherein different single emitters of the group emit different spectral ranges.

26. The device according to claim 25, wherein different single emitters of the group emit disjoined spectral ranges.

27. The device according to claim 24, comprising a plurality of groups of single emitters, wherein each of the groups is arranged upstream of a corresponding joint optics and at least one single emitter of each group is structured to emit non-coagulating light and at least one other emitter of each group is structured to emit coagulating light.

28. The device according to claim 25, wherein at least two other single emitters with differing emission spectral ranges are structured to emit coagulating light.

29. The device according to claim 24, wherein a plurality of groups of the single emitters emit white light with a non-coagulating illumination light power.

30. The device according to claim 24, wherein at least one proper subset of the single emitters emits white light with a non-coagulating illumination light power.

31. The device according to claim 29, wherein the single emitters are arrayed in a matrix-shaped arrangement of the single emitters and the white-light emitting single emitters are arranged in the outer fields of the single emitter matrix.

32. The device according claim 30, wherein the single emitters are arrayed in a matrix-shaped arrangement of the single emitters and the white-light emitting single emitters are arranged in outer fields of the single emitter matrix.

33. The device according to claim 22, wherein the optics comprise microlenses.

34. The device according to claim 22, wherein the optics are arranged in a coherent matrix.

35. The device according to claim 18, wherein the therapy beam path is free of mirrors, which are movable for scanning of the treatment region, and free of movable lenses, which are movable for scanning of the treatment region, and free of fiber-optic cables.

36. The device according to claim 18, further comprising a zoom optics arranged in the therapy beam path.

37. The device according to claim 22, wherein the single emitters and the downstream optics are structured such that a balance ratio of a distance between two adjacent surfaces in the treatment region and a diameter of one of the surfaces is between one and two.

38. The device according to claim 18, further comprising a control unit which successively activates different subsets of all single emitters.

39. The device according to claim 38, wherein the control unit successively activates disjoined subsets of all single emitters.

40. The device according to claim 38, wherein the control unit successively activates at least two elements in each subset.

41. The device according to claim 40, wherein the control unit successively activates congruent envelopes of said subset.

42. The device according claim 18, wherein the therapy beam path is coupled into the observation beam path of the ophthalmological device.

43. The device according claim 18, wherein the radiation source is energized by a battery, an accumulator or both the battery and the accumulator such that the device can be operated independently of a main power supply.

44. The device according to claim 18, wherein the single emitters are arranged exclusively along at least one straight line and a detection beam path projects respective surfaces of the treatment region onto at least one row of a plurality of detector elements.

45. A computer implemented method for operating a device comprising a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other, the method comprising:
activating a single emitter with non-coagulating radiation power in a group of single emitters which is jointly projected by a joint optics;
identifying of a signal for coagulation; and
activating at least one single emitter with coagulating radiation power in each of the groups.

46. A computer implemented method for operating a device comprising a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other, the method comprising:
activating a plurality of single emitters that irradiate along a first line with non-coagulating radiation power;
identifying a first signal for coagulation;
activating a plurality of single emitters that irradiate along the first line with coagulating radiation power; and
activating a plurality of single emitters that irradiate along a second line that is disjoined with the first line with non-coagulating radiation power;
identifying a second signal for coagulation;
activating a plurality of single emitters that irradiate along the second line with coagulating radiation power.

47. A computer readable data storage medium, comprising instructions that cause a computer operably coupled to a device comprising a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other, to perform a method comprising:
activating a single emitter with non-coagulating radiation power in a group of single emitters which is jointly projected by a joint optics;
identifying of a signal for coagulation;
activating at least one single emitter with coagulating radiation power in each of the groups.

48. A computer readable data storage medium, comprising instructions that cause a computer operably coupled to a device comprising a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other, to perform a method comprising:
activating a plurality of single emitters that irradiate along a first line with non-coagulating radiation power;
identifying a signal for coagulation;
activating a plurality of single emitters that irradiate along the first line with coagulating radiation power; and
activating a plurality of single emitters that irradiate along a second line that is disjoined with the first line with non-coagulating radiation power;
identifying a second signal for coagulation;
activating a plurality of single emitters that irradiate along the second line with coagulating radiation power.

49. A control unit operably coupled to a device comprising a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other, the control unit being programmed to perform a method comprising:
activating a single emitter with non-coagulating radiation power in a group of single emitters which is jointly projected by a joint optics;
identifying of a signal for coagulation;
activating at least one single emitter with coagulating radiation power in each of the groups.

50. A control unit operably coupled to a device comprising a radiation source that produces radiation for photocoagulation or phototherapy of tissue, including a retina of an eye;
a therapy beam path extending from the radiation source to a treatment region; and
an observation beam path;
wherein the radiation source comprises a plurality of disjoined single emitters and the therapy beam path projects at least respective surface segments of different single emitters simultaneously onto surfaces in the treatment region that are spaced apart from each other, the control unit being programmed to perform a method comprising:
activating a plurality of single emitters that irradiate along a first line with non-coagulating radiation power;
identifying a signal for coagulation;
activating a plurality of single emitters that irradiate along the first line with coagulating radiation power; and
activating a plurality of single emitters that irradiate along a second line that is disjoined with the first line with non-coagulating radiation power;
identifying a second signal for coagulation;
activating a plurality of single emitters that irradiate along the second line with coagulating radiation power.
